(19) United States
(12) Patent Application Publication
(10) Pub. No.: US 2011/0152634 A1
(43) Pub. Date: Jun. 23, 2011

(54) Measuring Human Biological Fluid Levels

(76) Inventor: Jeff Thew, Spokane Valley, WA (US)

(21) Appl. No.: 12/641,187

(22) Filed: Dec. 17, 2009

(51) Int. Cl. A61B 5/145; A61B 5/00

(52) U.S. Cl. 600/301; 600/365

(57) ABSTRACT

This disclosure describes measuring and analyzing a biological fluid level using a telecommunications device. Measuring the biological fluid level includes initiating a measurement and analysis application to activate measuring and analyzing the biological fluid level. The telecommunications device transmits a first signal to activate a measurement device placed on a human body. Once the biological fluid level is measured, the telecommunications device detects a second signal. Upon detecting the second signal, the biological fluid level is received and compared against a baseline range as predetermined by a user. Next, the measured biological fluid level and a symbol illustrating conditions of the level are presented on a user interface of the display of the telecommunications device.

### TECHNICAL FIELD

The subject matter of this disclosure relates to a way of measuring and analyzing human biological fluid levels, and more specifically, on a telecommunications device capable of functioning with a measurement device and a transmitter.

### BACKGROUND

Individuals may desire to monitor biological fluid levels for many reasons. The reasons for monitoring may include being at risk for health conditions to occur, such as heart attacks, strokes, epileptic seizures, diabetes, and desiring to know their biological fluid levels based in part on physical activity, dietary information, medication, or stress.

In a scenario, people diagnosed with diabetes often have a need to test their blood sugar or glucose levels regularly and frequently. The testing helps assess the effectiveness of diet, physical activity, and medication. Thus, the blood sugar or glucose levels measured help an individual identify what steps or strategies to take based on the blood sugar or glucose levels.

Typically, a traditional way of testing blood sugar or glucose levels involve cleaning one's finger with alcohol wipes, using a lancet (i.e., a spring-loaded device) to prick one's finger to put a drop of blood on a test strip, and then placing the strip into a meter that displays the blood sugar or glucose levels. The meters that record the information differ in a variety of features, such as a size of a display screen, a speed in recording the blood sugar or glucose levels, along with a size and a cost of the meter. However, most of the meters that are used traditionally tend to be rather large and bulky, and not very portable or convenient for the individual to carry to various locations.

Other problems with the traditional way of testing glucose levels are having to purchase wipes and strips at the store, which tend to be costly and pricking one's finger, which may cause pain and discomfort for the individual. Another problem is the inconvenience of carrying the alcohol wipes, the strips, the lancet, and the meter with the individual on a daily basis for continuous monitoring. There exists a need to measure human biological fluid levels.

### SUMMARY

This Summary introduces concepts with more description in the Detailed Description. This disclosure describes measuring and analyzing a biological fluid level using a telecommunications device. Measuring the biological fluid level includes initiating a measurement and analysis application to activate measuring and analyzing the biological fluid level. The telecommunications device transmits a first signal to activate a measurement device placed on a human body. Once the biological fluid level is measured, the telecommunications device detects a second signal. Upon detecting the second signal, the biological fluid level is received and compared against a baseline range as predetermined by a user. Next, the measured biological fluid level and a symbol illustrating conditions of the level are presented on a user interface of the display of the telecommunications device.

In an implementation, the process measures blood sugar or glucose levels using the telecommunications device as described above. Furthermore, the process may determine whether the measured blood sugar or glucose levels are within or outside a glucose range and alert the individual if outside the range by various alert mechanisms.

In another implementation, the process measures blood sugar or glucose levels using the telecommunications device as described above. Furthermore, the process may determine whether the measured blood sugar or glucose levels are within, below, or above the glucose range and provide recommendations for a course of action.

### BRIEF DESCRIPTION OF THE DRAWINGS

The detailed description is described with reference to the accompanying figures. In the figures, the use of the same reference numbers in different figures indicates similar or identical items. These drawings depict only illustrative examples of the invention and are not, therefore, to be considered to be limiting of its scope.

FIG. 1 is a schematic view showing an exemplary environment for measuring and analyzing biological fluid level using the telecommunications device.

FIG. 2 is a block diagram showing an exemplary telecommunications device for measuring and analyzing biological fluid levels.

FIG. 3 is a schematic showing an exemplary user interface of the telecommunications device with an exemplary measurement and analysis application.

FIG. 4 is a schematic showing the exemplary measurement and analysis application on the user interface of the telecommunications device, when a measured level is approximately the same as a baseline level or within a predetermined range.

FIG. 5 is a schematic showing the exemplary measurement and analysis application on the user interface of the telecommunications device, when the measured level is not the same as the baseline level or outside a predetermined range.

FIG. 6 is a schematic showing an exemplary measurement and analysis application on the user interface of the telecommunications device, when showing a chart.

FIG. 7 is a flowchart showing an exemplary process of measuring and analyzing blood sugar or glucose levels using the measurement and analysis application on the telecommunications device.

FIG. 8 is a flowchart showing an exemplary process of determining when the blood sugar or glucose levels are within or outside a predetermined range and sending alerts.

FIG. 9 is a flowchart showing an exemplary process of determining when the blood sugar or glucose levels are within or outside the predetermined range and providing recommendations for a course of action to take.

### DETAILED DESCRIPTION

As described above, conventional ways of testing biological fluid levels may not always provide convenient or effective ways of measuring levels. For example, in some instances, young children or adults that have been recently diagnosed with diabetes may not remember a baseline level nor understand a range that is predetermined by a health provider. Furthermore, conventional ways do not help individuals know what strategies to take based on the measured levels.

This disclosure describes various exemplary ways of measuring and analyzing biological fluid levels, for example, by utilizing a processing power of a processor on the telecommunications device to perform a variety of functions and to interact with many other applications. The processing power is superior to that provided by conventional meters for measuring fluid levels. The telecommunications device offer many other applications that provide valuable services in conjunction with the measured levels. With the capability of the processing power and interacting with the other applications, the measurement and analysis application described herein, may provide a variety of alerts when the readings are outside the range, may provide a course of action to take based on the readings, may send the readings that have been stored during a time period to a medical provider, and may receive a medical treatment plan from the medical provider. The terms, measured levels, readings, or received levels are used interchangeably to describe levels that have been measured by a measurement device and subsequently received by the telecommunications device.

Conventional measuring techniques, however, requires that an individual brings supplies such as alcohol swipes, strips, lancet, and meter, if he or she desires to measure their biological fluid levels. The lack of portability may be undesirable when, for example, engaged in daily activities away from home or travel. Having a measuring and analyzing application on the telecommunications device offer many possibilities, for example, by being able to measure biological fluid levels frequently, without having to carry supplies around, and without having to remember baseline or range fluid levels. Thus, it is desirable to have the measurement and analysis application on the telecommunications device, and provide the enhanced functionalities and operations. Furthermore, the measurement and analysis application may offer various functionalities, such as sending the readings of a young child and/or alerts over a network to a parent or the medical provider. The application may correlate the readings to dietary information, physical activity, or medication; and show trends and patterns with several readings over a time period. Furthermore, the application may provide a course of action to take based on the readings.

In some implementations, the measuring and analyzing application is used for individuals diagnosed with a condition or at risk for certain conditions to occur. However, in other implementations, the methods and devices of the present disclosure may be used to monitor several biological fluid levels in conjunction with dietary information, physical activity, and medication. For example, the measuring and analyzing application used together with other applications on the telecommunications device may allow an individual to differentiate the readings after eating a meal. This may be correlated that the blood sugar or glucose levels along with cholesterol levels were measured shortly after eating the meal within a time frame. Accordingly, the telecommunications device of the present disclosure is used to measure and to analyze the information, to send alerts, to show trends, and to provide a course of action in response to the readings.

In some implementations, the measurement and analysis application is used for individuals diagnosed with diabetes. The illustrations may apply to diabetes as examples, but could be applied to measure and to analyze other types of conditions or risks.

### Illustrative Environment

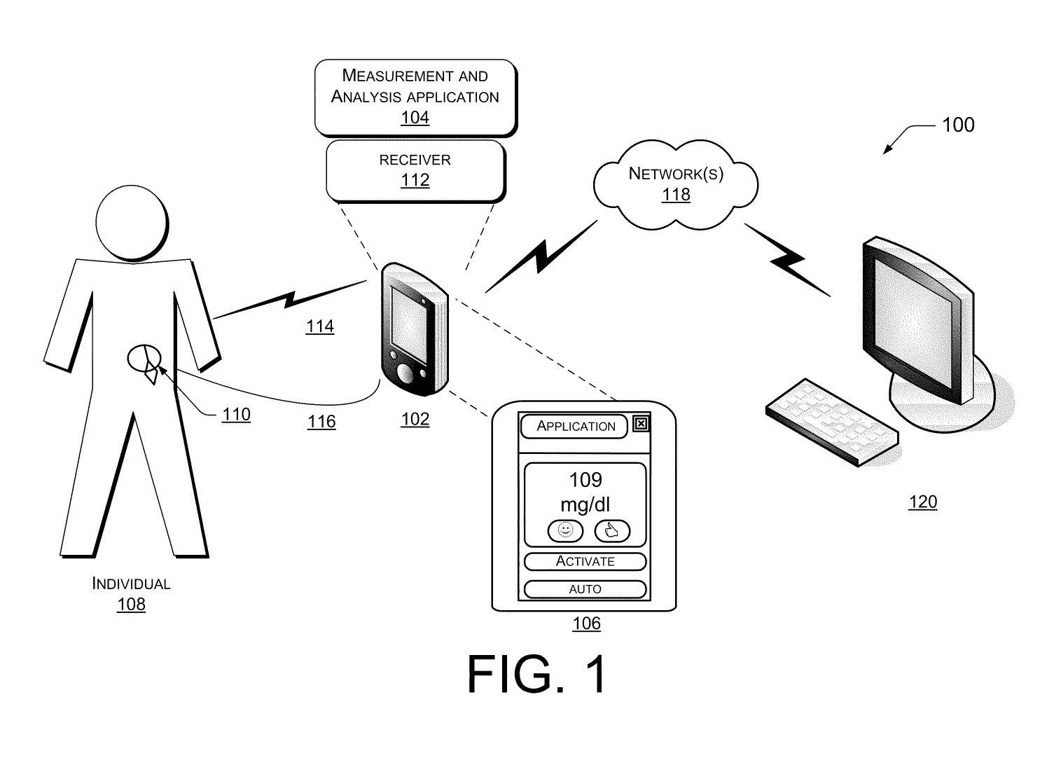

FIG. 1 is a schematic view showing an exemplary environment 100 for measuring and analyzing biological fluid levels using the telecommunications device 102. The telecommunications devices 102 that are suitable for use with the environment, include, but are not limited to, a cellular communication device, a smart communication device, a portable media player, a mobile communication device, a personal digital assistant, a laptop, and the like. A measurement and analysis application 104 is stored in memory of the telecommunications device 102 to provide a variety of functions and to interact with many other applications in storage. Shown in FIG. 1, the measuring and analyzing of biological fluid levels is implemented as an application program 104 operating on an operating system. In another implementation, the measuring and analyzing of biological fluid levels on the telecommunications device 102 may be provided as a service through a service provider, without downloading the application.

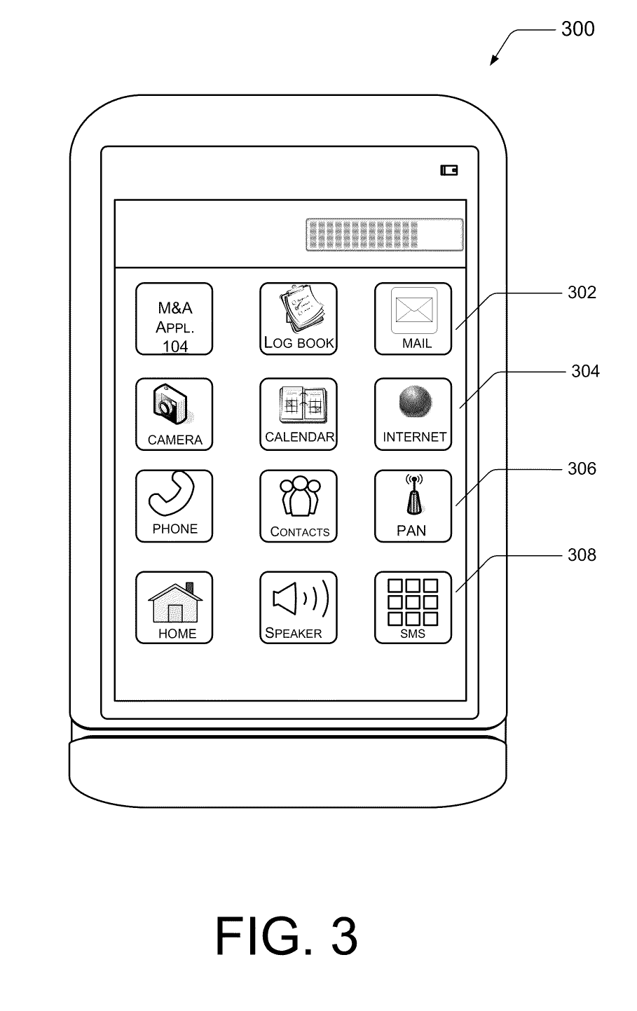

The telecommunications device 102 may include a display for a user interface 106 to access applications, view measured levels or readings, see trends or charts in measured levels or readings, and the like. The user interface 106 may include but is not limited to, receive input to access applications, to activate measuring the biological fluid levels, to view charts, to send readings via email, to receive a treatment plan based on the readings, and the like. In FIG. 1, the user interface 106 shows an exemplary display of a measured blood sugar or glucose levels at 109 gm/dl. A detailed discussion of the user interface 106 follows in FIGS. 3-6.

An individual 108 is shown wearing a measurement device connected to a transmitter 110. The measurement device may be formed integrally or coupled with an adhesive pad on one side and the transmitter 110 is located on the other side of the adhesive pad that is disposable. The measurement device may include, but is not limited to a probe, a sensor (i.e., a round, platinum-based miniature wire), a catheter, a needle, a cannula, a tube, or the like placed on a human body. The measurement device is placed on a portion of the human body, which is held in place by the adhesive pad. These devices are known in the art and are not discussed at length here. The biological fluids that may be collected include blood sugar or glucose, interstitial fluid, cholesterol, salvia, perspiration, heart rate, pulse, respiratory, and the like. The transmitter 110 is an electronic device to propagate an electromagnetic signal. The transmitter 110 may send the measured levels, readings, or received biological fluid levels to a receiver 112 on the telecommunications device 102 in a wireless network 114 or a wired interface 116.

A communication interface between the receiver 112 on the telecommunications device 102 and the transmitter 110 may be wireless 114 and/or wired 116. The wireless communication interface 114 may include, but is not limited to, Personal Area Network (PAN), IEEE 802.11x, AM/FM radio signals, wireless universal serial bus (USB), wireless local area network (LAN), or the like. The wired communication interface 116 may include, but is not limited to, controller-area network, recommended standard RS-232, universal serial bus, stereo wire, IEEE 1394 serial bus standard (FireWire) interfaces, or the like.

The telecommunications device 102 is capable of connecting to one or more networks, as represented by network 118. The network 118 is representative of any one or combination of multiple different types of networks, interconnected with each other and functioning as a single large network (e.g., the circuit-switched telephone networks or IP-based packet-switch networks). The network 118 may also include wire-based networks (e.g., cable) and wireless networks (e.g., cellular, satellite, etc.). The network 118 may use any number of protocols and configurations to enable the telecommunications device 102 to access other devices and resources. The network may include but is not limited to, a Personal Area Network (PAN), a Local Area Network (LAN), a wireless Wide Area Network (WAN), a Metropolitan Area Network (MAN), a Virtual Private Network (VPN), and a Campus Area Network (CAN).

The network 118 may include several types of circuit-switched telephone networks, such as Public Switched Telephone Network (PSTN) and packet-switched networks. For example, the network 118 may include Global System for Mobile communication (GSM)/UMTS cellular network, a Code Division Multiple Access (CDMA) cellular network, other types of circuit-switched networks and/or the Internet.

The telecommunications device 102 may communicate with one or more computing devices 120 via the network 118. In an implementation, the computing device 120 may belong to a parent that receives notifications or alerts when a child with juvenile diabetes receives an abnormally high reading. In another implementation, the computing device 120 may belong to a medical provider that receives notifications or alerts of abnormally high readings and/or a log of readings taken during a predetermined time period. For example, the measurement and analysis application 104 may automatically take readings of the individual 108 six or more times a day during a 3 month time period, send the readings to the medical provider, and based on the readings, receive a medical treatment plan from the medical provider. In yet another implementation, the computing device may belong to the individual to be able to print out the readings or the measured levels.

### Exemplary Telecommunications Device

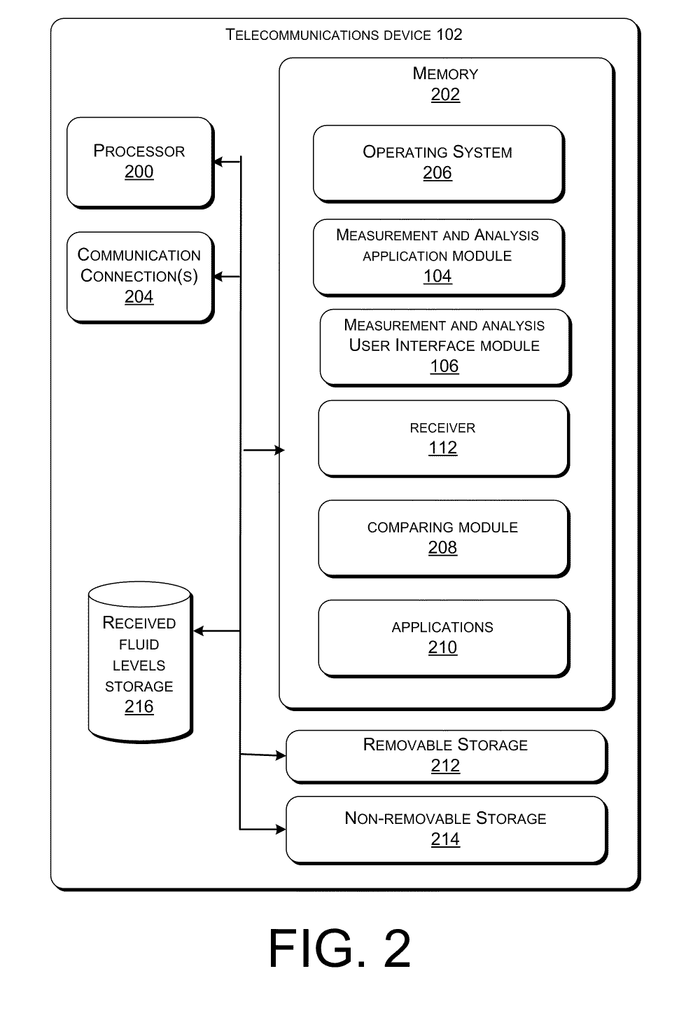

FIG. 2 is a block diagram showing an exemplary telecommunications device for measuring and analyzing biological fluid levels of FIG. 1. As shown, the telecommunications device 102 includes a processor 200, a memory 202, and one or more communication interface(s) 204. The communication interface 204 may provide a communicative connection via a wired or wireless communication interface to the transmitter 110 or to the other computing device 122. Thus, the communication interface 204 allows the telecommunications device 102 to communicate over the wired communication interface 114, the wireless communication interface 116, and wired or wireless on the network(s) 118. For example, the telecommunications device 102 may include the receiver 112 to receive readings from the transmitter 110, such as opening a communication channel to await a transfer of the readings.

The memory 202 may include an operating system 206, a module for the measurement and analysis application 104, and a module for the user interface (UI) 106 that, when executed on the processor 200, collectively facilitate presentation of the user interface 106 on the display of the telecommunications device 102. The telecommunications device 102 of the individual 108 may, in some implementations, visually present readings of the biological fluid levels. This visual representation of the received biological fluid levels allows the individual 108 to visually verify whether their levels are low, high, or within a baseline or a predetermined range. Furthermore, in some implementations, the user interface 106 may visually present a calendar, a logbook for dietary information, physical activity to correspond to the readings. In another implementation, visual representation of the readings is presented in a graph that allows the individual 108 to visually verify that the readings are within a predetermined range. Details of several illustrative user interfaces that may be presented are described in FIGS. 3-6.

The memory 202 may include a comparing module 208 to provide analysis of the level. The application 104 compares the biological fluid level or reading taken to a predetermined baseline identified by a user. In another implementation, the comparing functionality may be a part of the measurement and analysis application 104. The user may be the individual, a medical provider, a medical consultant, a doctor, an endocrinologist, a medical specialist, and the like. The individual 108 may set the predetermined baseline on the measurement and analysis application 104 or alternatively, the medical provider may set the baseline if access is granted or if the medical provider directly sends a treatment plan to the telecommunications device 102.

The comparing module 208 may also identify a symbol and/or an indicator to show in parallel with the reading on the display. The desired symbol and/or indicator may be determined in advance by the individual 108 and/or the medical provider. In an implementation, the comparing module 208 may determine the symbol to be used based on comparing the received glucose level to the baseline glucose level or a predetermined range. When the received glucose is approximately about the same number as the baseline glucose level, the symbol may include a smiley face, a thumbs up, or a color indicator. The color indicator may range from green and yellow, depending on the received glucose level. Furthermore, when the received glucose level is approximately 15 mg/dl less than or approximately 15 mg/dl greater than the baseline glucose level, the symbol may include a frown face, a thumbs down, or the color indicator of red. Again, the difference between the received glucose level and the baseline glucose level may be customized for each individual 108, as determined by the medical provider, if given access. Settings may be performed by the individual, the medical provider, or the parent for a child.

The comparing module 208 may also determine if the biological fluid level or reading is within a predetermined range. In an implementation, the comparing module 208 may determine whether the received glucose level is outside or within a glucose range, predetermined by the user. In response to the received glucose level being within the glucose range, the measurement and analysis application 104 may store the received glucose level in storage of the telecommunications device 102. However, in response to the received glucose level being outside the glucose range, the measurement and analysis application 104 may generate an alert on the telecommunications device 102 to vibrate, to play an audible sound on a speaker, to vibrate and to play the audible sound, to send an email to an identified address, to send a text message to an identified cellular number, or to send the email to the identified address and to send the text message to the identified cellular number. One or more of the identified combinations may occur, depending on how the alert or warning conditions are set up and customized for each individual. These are described as exemplary alert mechanisms. Next, the measurement and analysis application 104 may store the received glucose level in storage of the telecommunications device 102.

In some instances, a child may be diagnosed with juvenile diabetes or a woman diagnosed with pregnancy diabetes, the readings may be sent via email and/or text to the parent or medical provider, respectively. In another instance, an individual may be newly diagnosed with diabetes and not aware of the baseline or range. In this instance, the measurement and analysis application 104 may be set to measure readings on the individual four, six or more times daily, and to send an alert via vibrating and playing a voice message. Furthermore, the individual may take a course of action that has been prescribed for him.

The memory 202 may include one or more other applications 210 for implementing a part of the biological fluid level services or measurement and analysis application 104. The other applications 210 offer various other functionalities used in conjunction with the measurement and analysis application 104. The other applications 210 include, but is not limited to, an email application, a logbook, a calendar application, a text messaging application, a word processing application, a spreadsheet application, a media player application, a camera, and the like.

The telecommunications device 102 may also include additional removable storage 212 and/or non-removable storage 214. Any memory described herein may include volatile memory (such as RAM), nonvolatile memory, removable memory, and/or non-removable memory, implemented in any method or technology for storage of information, such as computer-readable instructions, data structures, applications, program modules, emails, and/or other content. In addition, any of the processors described herein may include onboard memory in addition to or instead of the memory shown in the figures. The memory may include storage media such as, but not limited to, random access memory (RAM), read only memory (ROM), flash memory, optical storage, magnetic disk storage or other magnetic storage devices, or any other medium which can be used to store the desired information and which can be accessed by the respective systems and devices.

The memory 202 may also include a received fluid levels storage 216 for locally storing one or more readings. Storing the readings on the received fluid levels storage 216 offers the individual 108 accessibility to the readings, if there is no network service available. Once the readings have been stored, a file may be created. The file may be saved on a memory stick to take to another computing device or a printer, attached to an email for sharing, or transferred to another computing device or printer.

The telecommunications device 102 may also include one or more known input device(s), such as a keyboard, mouse, pen, voice input device, touch input device, etc., and output device(s), such as a display, speakers, printer, etc. All these devices are well known in the art and are not discussed at length here.

While the telecommunications device 102 is shown and described as having certain hardware and software modules, it should be understood that all modules may be implemented as appropriate in hardware, software, firmware, or combinations thereof. If implemented by software, the software may reside on memory associated with any component of the telecommunications device 102, standalone memory provided in connection with the telecommunications device 102, a remote memory storage device, removable/nonremovable memory, a combination of the foregoing, or any other combination of one or more processor-readable media. While the telecommunications device 102 is shown as having certain modules, it should be understood that in some implementations, one or more of the modules could be combined or omitted entirely.

### Exemplary Telecommuications Device User Interface

FIGS. 3-6 illustrate exemplary user interfaces that may be presented on the telecommunications device 102 of the individual 108. There are various options that may be available on the telecommunications device 102, such as large touch-screen interfaces, alpha-numeric keypads, and other forms of input and display. The user interfaces described below are illustrative of user interfaces usable with a touch-screen interface. However, other user interfaces may be used if other types of telecommunications devices are used.

Blood sugar or glucose level is used as an example of the human biological fluid being used in these illustrations or implementations. However, it should be understood that blood sugar or glucose level contained in this disclosure need not be the only type of measurements described. The user interface and flowcharts are described in the context of blood sugar or glucose levels. However, the user interfaces and processes may be implemented measuring and analyzing other human biological fluid levels, alone or in combination.

FIG. 3 is a schematic showing an exemplary user interface 300 with the measurement and analysis application 104. Shown here, the individual 108 may initiate the measurement and analysis application 104 by receiving input to activate functionality for the measurement and analysis application 104. In this example, the user interface 300 includes the applications that are usable by the individual 108. The user interface 300 may include the measurement and analysis application 104, a log book, and an email application, as shown along row 302. Selection of the measurement and analysis application 104 opens the application to measure glucose levels, to access readings on a chart, to set conditions, ranges, alerts, to enter information on the logbook, and the like. As previously mentioned, the individual 108 may log dietary information, physical activity, or medication usage in the logbook shown on the user interface 300 or access the logbook within the measurement and analysis application 104, and send the readings with this information to the medical provider, via email.

Other applications may include a camera, a calendar, and access to the internet, as shown along row 304. As previously mentioned, the calendar may correspond to the readings taken and received at certain times and days of the month. Alternatively, the calendar entries may include a date and time along with a short description of an appointment or an event. This calendar information may provide additional information to explain why blood glucose levels may be out of range, such as after a meal, after a holiday, or on a weekend. The internet provides a way for the readings on the telecommunications device 102 to communicate over the network 118 with another computing device 120, such as the computing device 120 belonging to the medical provider and/or the parent.

The user interface 300 also includes additional applications allowing the individual 108 to access a phone, a list of contacts stored in memory of the telecommunications device 102, and a personal area network (e.g., Bluetooth), as shown along row 306. The personal area network may be one form of a communication connection from the telecommunications device 102 to the transmitter 110.

The user interface 300 shows how the individual 108 may move from one screen to another, such as home, access a speaker, or send a text message, as indicated on row 308. The text message may be preprogrammed to send alerts or warnings when the measured levels are outside the range to the parent, for the young patient or the medical provider, for the patient.

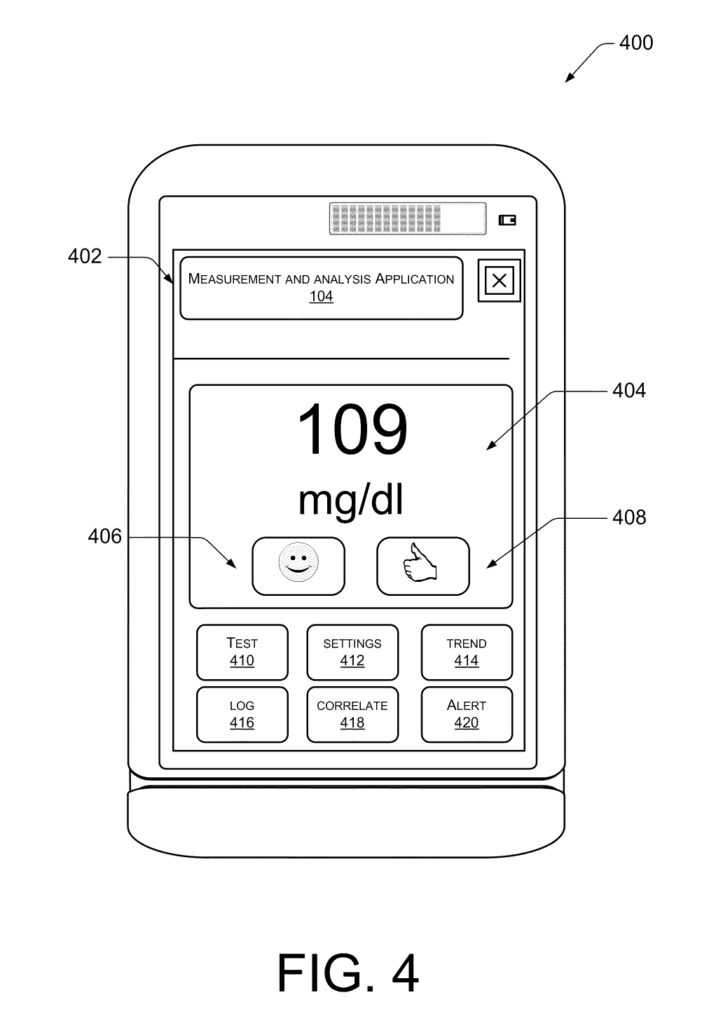

FIG. 4 shows an exemplary user interface 400 when the received level or the measured level is approximately the same as the baseline level or within the predetermined range. In FIG. 4, the user interface 400 includes a title bar 402 to illustrate a current application that is being used, the measurement and analysis application 104. In the illustrated example, the measured or received blood sugar or glucose level of 109 mg/dl at 404. Milligrams (mg)/deciliter (dl) is a traditional unit for measuring glucose level. In some instances, the measurements may be shown in a world standard unit for measuring blood sugar in mmol/l, which is millimoles/liter. The user interface shows the measured glucose level at 404 along with the symbol of “a smiley face” at 406 and an indication of “thumbs up” at 408. A single symbol or the symbol in combination with other symbols or indications may be used to show the measured level at 404 is approximately the same as the baseline level or is within a predetermined range.

The predetermined range may be identified by a medical provider and set by the medical provider and/or the individual 108. The terms predetermined range and normal range may be used interchangeably to indicate the range to maintain for the individual. By maintaining levels within this range, the individual 108 may prevent or delay the onset of complications. For example, if the individual 108 has diabetes, their blood sugar or glucose levels need to be monitored very frequently to prevent complications that may occur. Some of the complications may include glaucoma, neuropathy damage, skin disorders or infections, heart disease, high blood pressure, kidney disease, peripheral arterial disease, and the like. If the glucose level is high, it may cause hyperglycemia. On the other hand, if the glucose level is low, it may lead to hypoglycemia. Glucose levels may vary from 80 mg/dl to 110 mg/dl, except shortly after eating. Before meals, the glucose levels may vary from 90 mg/dl to 130 mg/dl and after meals, as high as 180 mg/dl. The treatment plan may be described for each individual.

Other indications may include a green, a yellow, or a red color. The green color indicates the reading is approximately the same as the baseline level or within the predetermined range. The yellow color indicates the reading is slightly higher than the baseline or slightly close to being outside the predetermined range. In this instance, a course of action may be recommended. The red color indicates the reading is higher than the baseline level and is outside the predetermined range. In this instance with the red color, a course of action is recommended.

In another implementation, the received or measured level may be announced on the speaker of the telecommunications device 102. A text-to-speech (TTS) system converts normal language text into speech using one or more known techniques. A speech synthesis module creates audible speech from pieces of recorded speech stored in a database. In particular, the speech synthesis module may in some instances present the received or measured level in an audible manner. For example, the individual 108 may be driving a vehicle, when their blood sugar level or glucose level is measured. Since their telecommunications device 102 may not be readily accessible to view the received or the measured level, presenting the reading in this audible manner allows the individual 108 to take a course of action, if needed. In yet another implementation, there may be a second audible presentation that the received or the measured level is within the range.

The user interface 400 may show various fields that the individual 108 may activate. The individual 108 may include a “Test” 410 field to initiate or to activate measuring and analyzing biological levels for particular sensor(s). As mentioned, there may be multiple sensors placed on different parts of the body to measure different body levels. Thus, this may activate certain sensors to measure various biological fluid levels. For example, the individual 108 may desire to measure the blood sugar or glucose level on demand, since the individual 108 may want to know their blood sugar or glucose level at the moment, rather than receiving the readings during the predetermined time periods.

The user interface 400 may include a “Settings” 412 field to set up guidelines or when to automatically measure, how often to measure, which biological fluid levels to measure, setting a medical treatment plan, setting course of actions to take based on levels, when to send text or email messages when levels are outside ranges, when to alert individuals about levels that are outside ranges, which biological fluid levels to correspond to in charts or data, and the like.

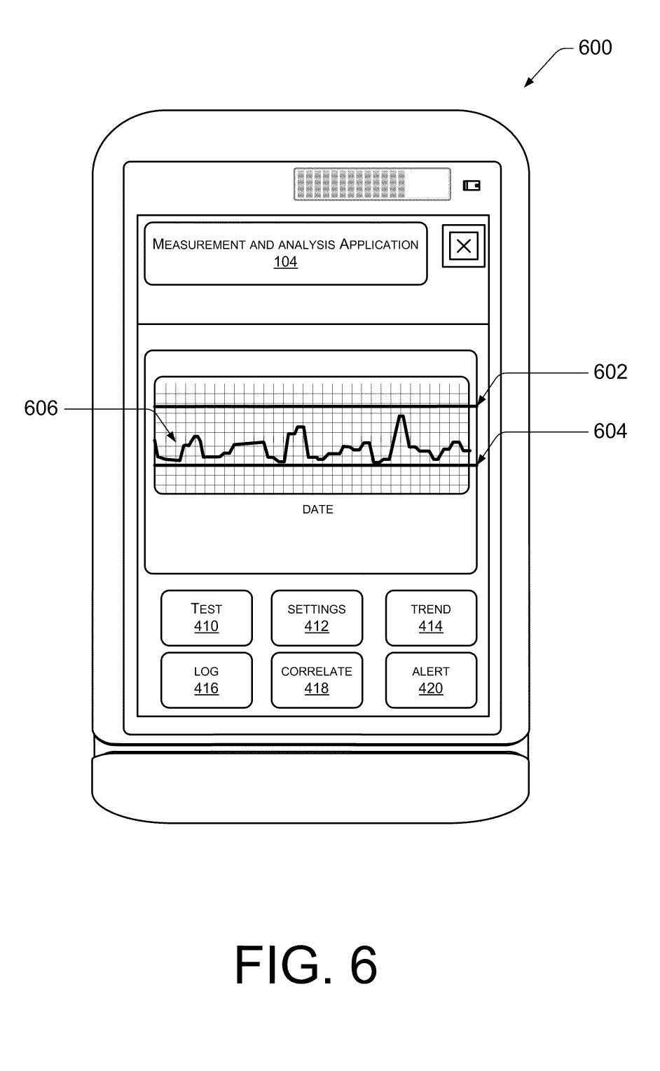

The user interface 400 may include a “Trends” 414 field to show graphs of the one or more received or measured levels during certain time periods. For instance, the individual 108 may be newly diagnosed with diabetes and have a need to know their blood sugar levels by being tested at least eight times or more per day. An exemplary graph is shown in FIG. 6.

The user interface 400 may include a “Log” 416 field to access or to provide entries of one's daily readings or daily activities in a logbook. The readings may be shown individually, shown based on a date, and/or shown based on a grouping. Alternatively, there may be a separate logbook function on the user interface 300 to access the logbook. The daily activities may show an entry for the individual 108 attending a birthday party, the blood sugar level is expected to be higher than normal due to having cake and ice cream available. This helps identify dietary information, physical activity, and medication dosage to the received levels.

Alternatively, the individual 108 may associate the readings with calendar information into the logbook functionality. Any of the functionality may be combined, using the reading level with the calendar and/or the logbook.

The user interface 400 may include a “Correlate” 418 field to correspond different biological readings from various parts of the human body. For example, the biological readings are performed by using different sensors to measure glucose, respiratory, and/or cholesterol levels. For example, there may be readings for the individual 108 with lower respiratory and blood sugar levels than normal, but a higher cholesterol level than the normal level. The measurements may have occurred when the individual 108 was asleep. This further helps identify dietary information, physical activity, and medication dosage to the received levels.

The user interface 400 may include a “Alert” 420 field to set up guidelines or when to automatically send alerts, how often to send alerts, which type of alert mechanisms, when to disable alerts, when to send text or email messages when levels are outside ranges, when to alert individuals about levels that are outside ranges, and the like.

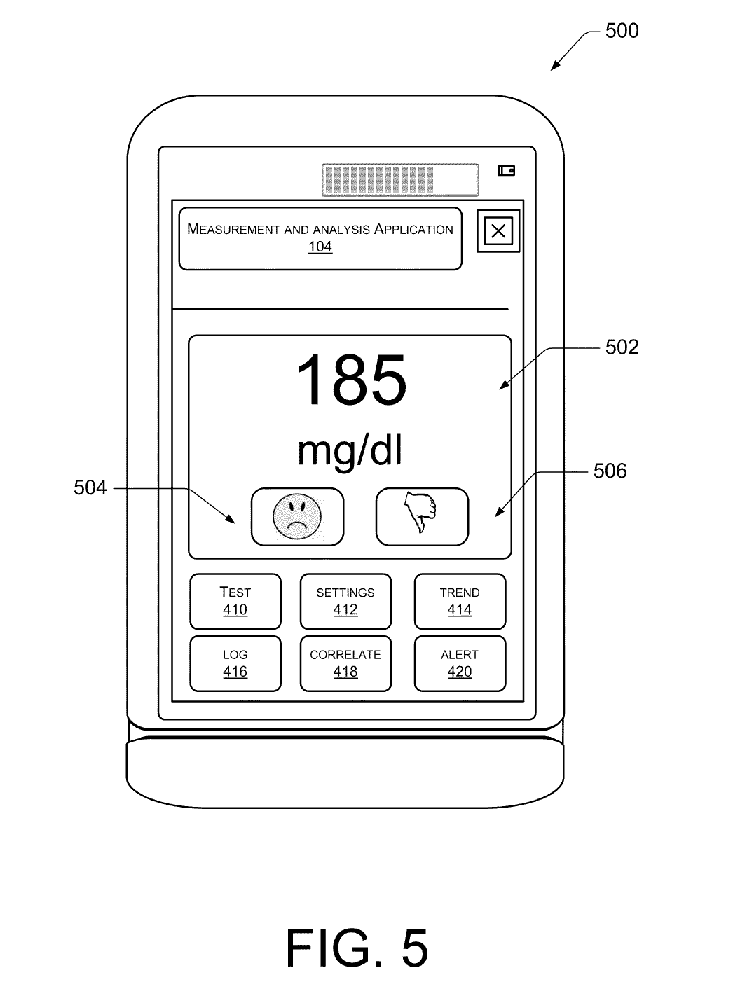

FIG. 5 shows an exemplary user interface 500 when the received level or the measured level is below or above the baseline level or outside the predetermined range. In the illustrated example, the measured or received blood sugar or glucose level of 185 mg/dl is shown at 502 along with the symbol of “a frown or unhappy face” at 504 and an indication of “thumbs down” at 506. A single symbol or in combination with another symbol or indication may be used to show the measured level at 502 is lower or higher than the baseline level. In an example, the blood sugar or glucose level of 75 mg/dl may be of concern, of being towards hypoglycemia.

As mentioned previously, the speech synthesis module may in some instances present the received or measured level in the audible manner. For example, the individual 108 may be driving a vehicle, when their blood sugar level or glucose level is measured. Since their telecommunications device 102 may not be readily accessible to view the received or the measured level, presenting the reading in this audible manner allows the individual 108 to take a course of action, if needed. In another implementation, the course of action may also be presented verbally, such to eat or drink carbohydrate when the measured level is low. In yet another implementation, there may be a second audible presentation that the received or the measured level is outside the range.

FIG. 6 shows an exemplary user interface 600 of received blood sugar or glucose levels in a chart. The medical provider or the individual 108 may set predetermined ranges based on the individual's health condition. The upper range or high blood sugar or glucose level is indicated as a dark line at 602. The lower range or low blood sugar or glucose level is indicated at 604. The measured blood sugar or glucose levels are shown at 606. In this example, the vertical axis is the blood sugar or glucose level given in mg/dl and the horizontal axis is shown as a time period or date that the blood sugar or glucose level is measured. For example, the chart may show trends over time, ranging from time periods of one day, a week, a month, or a three month period. In yet another implementation, the measurement and analysis application 104 may be automatically set to save the received glucose levels for the predetermined time and automatically send the saved glucose levels to the medical provider or to a home computing device. In response to the received glucose levels transmitted to the medical provider, it may provide and send a medical treatment plan to the telecommunications device 102. Access to the internet 118 offers many conveniences, such as communicating with the medical provider and sending to the home computing device for printing out the chart.

### Exemplary Processes

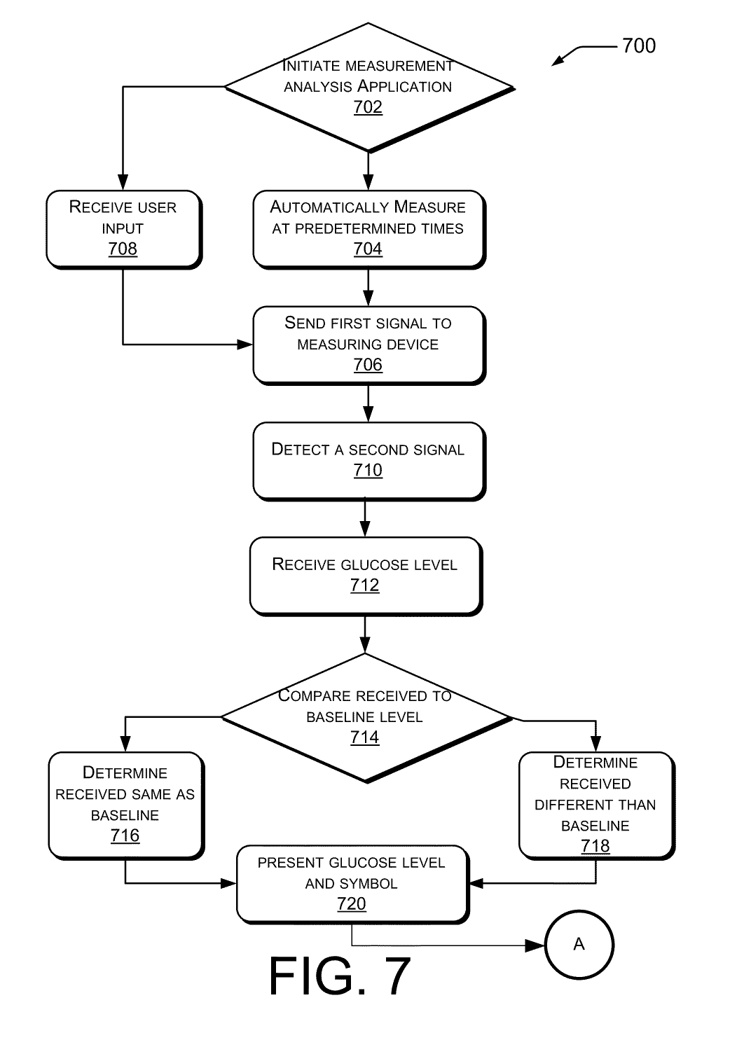

FIGS. 7-9 are flowcharts showing exemplary processes for measuring and analyzing blood sugar or glucose levels. The processes are illustrated as a collection of blocks in logical flowcharts, which represent a sequence of operations that can be implemented in hardware, software, or a combination. However, it should be understood that certain acts in each process contained in this disclosure need not be performed in the order described, may be modified, and/or may be omitted entirely, depending on the circumstances. For discussion purposes, the processes are described with reference to the computing environment 100 shown in FIG. 1, the telecommunications device 102 shown in FIG. 2, and the user interfaces shown in FIGS. 3-6. However, the processes may be performed using different architectures and devices. Moreover, the architectures and devices described herein may be used to perform different processes, such as measuring and analyzing other human biological fluid levels.

FIG. 7 is a flowchart showing an exemplary process 700 of measuring and analyzing blood sugar or glucose levels using the measurement and analysis application 104 on the telecommunications device 102. At block 702, the measurement and analysis application 104 is initiated to measure the glucose level. At block 704, the process 700 may be set to measure glucose levels automatically at predetermined times. For example, the individual may receive a treatment plan from the medical provider to test their glucose level at least eight times a day. In this instance, it would be more convenient to set up the predetermined times to automatically activate measuring the individual's glucose levels, on the measurement and analysis application 104. At block 706, the process 700 transmits a first signal through the transmitter 110 to activate a measurement device placed on a human body to measure the glucose level.

Returning to block 702, the process 700 may move along the left side to receive input on the user interface 708 to initiate the measurement and analysis application 104. In this example, the individual 108 may manually activate the measurement device to assess the effectiveness of their diet, exercise, or medication by testing the concentration of glucose in their body, shortly after eating, exercising, or taking medication. From here, the process 700 transmits the first signal to the measurement device, block 706. The measurement device tests the glucose level of the individual 108.

Upon completion of measuring the glucose level, the receiver 112 detects a second signal received from the transmitter 110 connected to the measurement device, block 710. The receiver 112 receives the measured glucose level from the measurement device, at block 712. The process 700 compares the received glucose level to the baseline glucose level predetermined by a user (e.g., medical provider, individual, and the like) at block 714. The baseline glucose level is identified as the level before eating meals, since after eating a meal, the glucose levels tend to go higher. If, at block 714, the received glucose levels is about the same as or approximately close to the baseline level, the process moves to the left side to block 716. In some instances, the settings for diabetes may range from about 80 mg/dl to 110 mg/dl, 90 mg/dl to 120 mg/dl, and 95 mg/dl to 125 mg/dl. Alternatively, the settings may be set to be approximately 10-30 mg/dl less than or greater than the predetermined baseline level. After the comparison, the symbol or the indication associated with the glucose level that is about the same or the glucose level that is approximately close to the baseline level, is shown with the reading at block 718. The process 700 visually presents the received glucose level along with the symbol, the indication, or both.

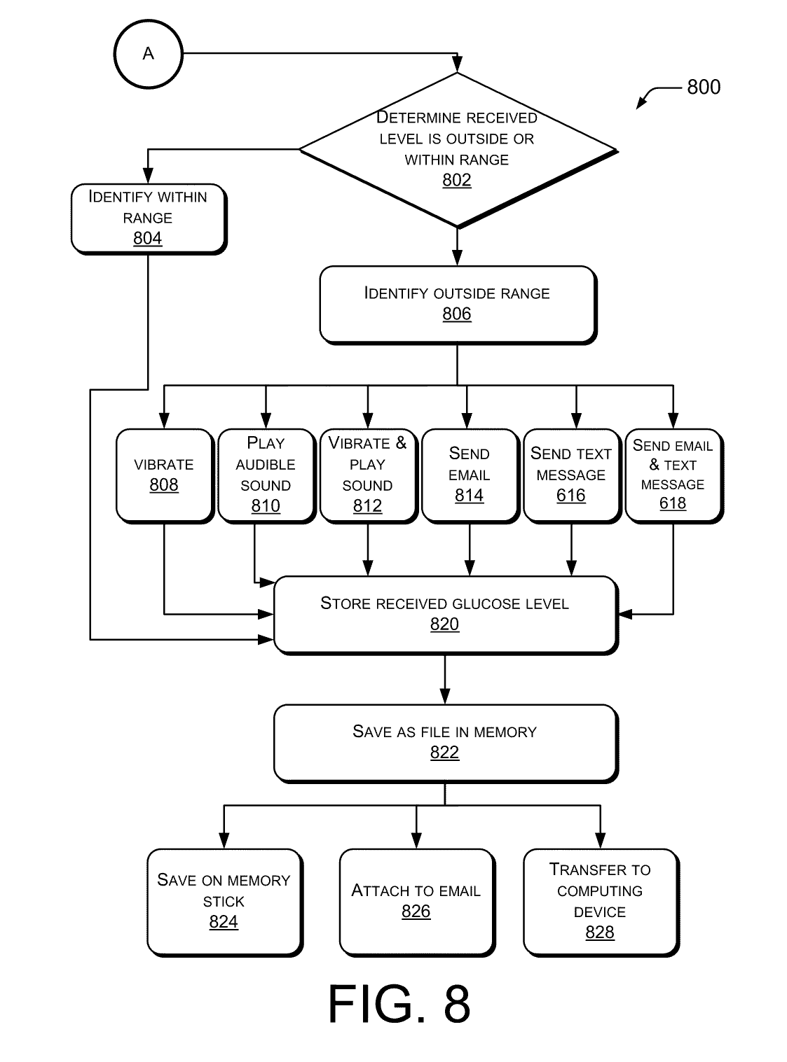

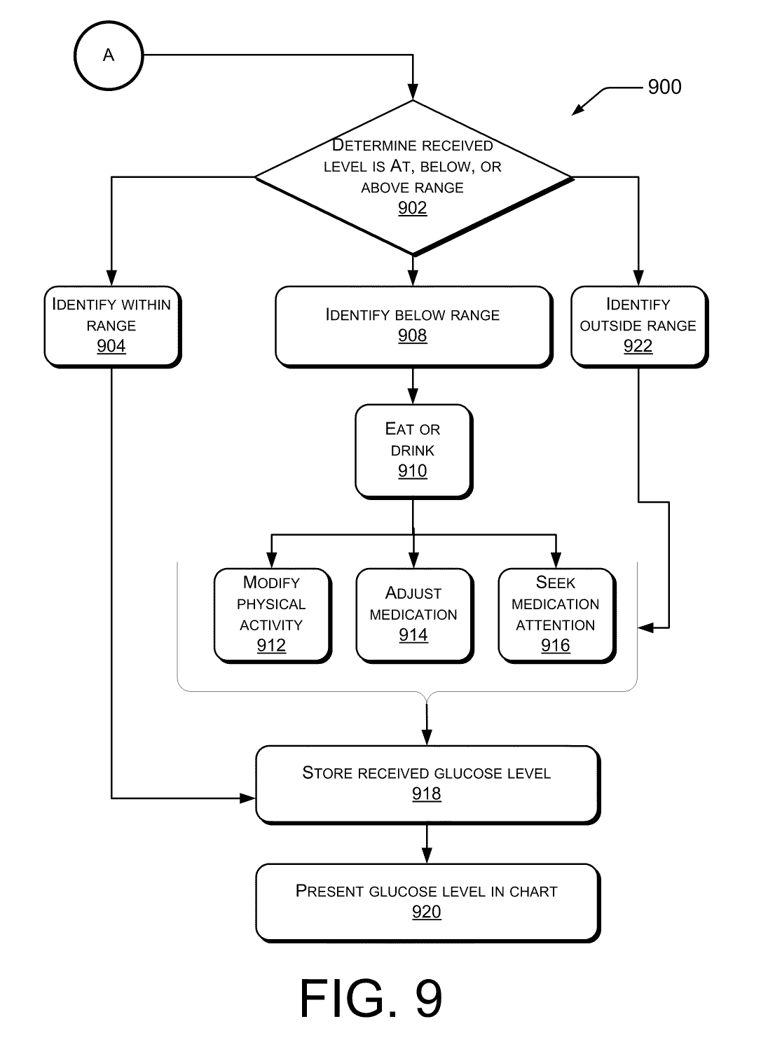

Returning to block 714, the process 700 compares the received glucose level to the baseline glucose level. If, at block 714, the received glucose level is lower or higher than the predetermined baseline level, the process moves along the right side to block 720. In some instances, the baseline settings may range from about 80 mg/dl to 110 mg/dl, 90 mg/dl to 120 mg/dl, and 95 mg/dl to 125 mg/dl. After the comparison, the symbol or the indication associated with glucose level that is lower or higher than the baseline level, is shown with the reading at block 718. The process 700 visually presents the received glucose level along with the symbol, the indication, or both. The process 700 may continue to FIG. 8 and/or FIG. 9.

FIG. 8 is a flowchart showing an exemplary process of determining when the blood sugar or glucose levels are within or outside the predetermined range and sending alerts or notifications. At block 802, the process determines whether the received glucose level is within or outside the range. The ranges for diabetes may be predetermined to be 80 mg/dl to 110 mg/dl, 90 mg/dl to 120 mg/dl, and 95 mg/dl to 125 mg/dl. If, at block 804, the received glucose level is approximately about the same number as the baseline glucose level, the symbol is the smiley face, the thumbs up, or the color indicator. One, two, or three symbols or indications may be used. As mentioned, the process may play an audible sound. The process 800 proceeds to store the received glucose level in memory of the telecommunications device 102, at block 820.

Returning to block 802, if at block 802, the received glucose level is outside the predetermined range, being approximately lower or higher than the baseline glucose level 806, the process associates the symbols or color indications. For measured glucose levels that are outside the range 806, the frown face, the thumbs down, or the yellow or red color indicator may be presented with the measured level. One, two, or three symbols or indications may be used.

In response to the received glucose level being outside the glucose range at block 806, the process 800 may generate an alert, a notification, or an alarm to occur on the telecommunications device 102. In block 808, the telecommunications device 102 may be set to vibrate to alert the individual 108 of their measured glucose level being outside the range. In block 810, the telecommunications device 102 may play an audible sound such as a tone, music, or announce the measured glucose number. At block 812, the telecommunications device 102 may be set to vibrate and to play the audible sound.

In response to the glucose level being outside the glucose range, the telecommunications device 102 may be set to send an email to an identified address at block 814, to send a text message to an identified cellular number at block 816, or to send the email to the identified address and to send the text message to the identified cellular number at block 818. Additionally or alternatively, the e-mail and text messages may be sent to more than one address or cellular number.

The alerts or notifications described above may be particularly useful when the glucose level is being measured for a young child or a teenager, who may have juvenile diabetes or for an adult who has insulin-dependent diabetes. Since these individuals tend to need insulin to maintain their glucose level, the alerts may be sent to the medical provider and/or the parent. In implementations, the individual may choose to turn off the alert notification, may provide input by acknowledging the alert has been received, and may continually receive the alert until the individual deactivates it, just for the measured level reading at a current session. Future readings would activate the alerts, if deactivated.

The telecommunications device 102 may be set to store the received glucose level in memory of the telecommunications device 102 at block 820. In an implementation, the received glucose level may be saved as a file in memory of the telecommunications device 102. Additionally or alternatively, the file saved on the telecommunications device 102 may be saved on a memory stick 824, attached to an email 826, or transferred to a computing device 828 by connecting the telecommunications device 102 to the computing device. These implementations offer the individual 108 various ways to share the received glucose levels with the medical provider or to print the measured glucose charts on a printer. As mentioned above, in response to the received glucose levels being transmitted to the medical provider in the email, the individual may receive a medical treatment plan from the medical provider.

FIG. 9 is a flowchart showing an exemplary process 900 of determining when the blood sugar or glucose levels are within or outside the predetermined range and providing recommendations for a course of action to take. The process 900 may continue from 700 or used in combination with 700 and 800. At block 902, the process determines whether the received glucose level is within, below, or above the glucose range predetermined by the user. The medical provider may provide the glucose range to the individual, in a medical treatment plan sent electronically or in a hard copy. If the range is identified in the hard copy, the individual 108 may set the range on the telecommunications device 102.

From block 902 proceeding along the left side, the received glucose level may be within the glucose level range 904. The process 900 may provide a recommendation for a course of action to take: continue physical activity or meal without any adjustments 906.

From block 902, process may proceed to the received glucose level may be below the glucose range 908. The process 900 may provide a recommendation for a course of action to take: to eat or to drink a fast-acting carbohydrate at block 910, to modify physical activity at block 912, to adjust medication at block 914, and/or to seek medical attention at block 916. Other courses of actions may be provided, depending on the medical condition and the human biological fluid level.

The process moves to store the received glucose level information at block 918 and presents the glucose level in a chart at block 920.

Returning to block 902, if the glucose level is outside the range, the process moves along the right side of the figure to block 922. In response to the received glucose level being above the glucose range, the process provides the recommendations for the course of action to take: to modify physical activity 912, to take medication 914, and/or to seek medical attention 916.

It is desirable to test or to monitor the individual's biological fluid levels or glucose levels to identify strategies based on the levels. Part of the process in managing diabetes also includes meal planning, planned physical activity, and learning how to handle the low and high blood sugar levels. Thus, this ongoing process of testing and analysis of biological fluid levels and glucose levels should be managed daily.

As previously mentioned, the measurement and analysis application 104 may be used for measuring and analyzing at least two or more biological fluid levels for different types of evaluation and associating the two or more received biological fluid levels with a physical activity, dietary information, or medication information based in part at least from a calendar or a logbook. The application 104, in response to the physical activity, dietary information, or medication information, determines whether the received biological levels are below or above a biological fluid level range predetermined by a user. Based at least to the received biological fluid levels being below the biological fluid range, provide a recommendation for a course of action to take: to modify physical activity, to adjust medication, or to seek medical attention. Based at least to the received biological fluid levels being above the biological fluid range, provide the recommendation for the course of action to take: to adjust dietary selections, to modify physical activity, to take medication, or to seek medical attention. Next, the application may store the received biological fluid levels in memory of the telecommunications device and present the information to the individual.

Any of the acts of any of the methods described herein may be implemented at least partially by a processor or other electronic device based on instructions stored on one or more processor-readable media. By way of example, and not limitation, processor-readable media may comprise volatile and nonvolatile, removable and non-removable media implemented in any method or technology for storage of information such as processor-readable instructions, data structures, program modules or other data. Processor-readable media includes, but is not limited to, RAM, ROM, electrically erasable programmable ROM (EEPROM), flash memory or other memory technology, compact disk-ROM (CD-ROM), digital versatile disks (DVD) or other optical storage, magnetic cassettes, magnetic tape, magnetic disk storage or other magnetic storage devices, or any other medium which can be used to store the desired information. Combinations of any of the above should also be included within the scope of processor-readable media.

### Conclusion

Although the subject matter has been described in language specific to structural features and/or methodological acts, it is to be understood that the subject matter defined in the appended claims is not necessarily limited to the specific features or acts described. Based on the teachings of the present disclosure, a variety of alternate embodiments may be conceived, and the present disclosure is not limited to the particular embodiments described herein and shown in the accompanying figures. Rather, the specific features and acts are disclosed as illustrative examples.

What is claimed is:

1. A method comprising:
under control of at least partially by a telecommunications device configured with executable instructions:
presenting a user interface on a display of the telecommunications device, the user interface including a measurement and analysis application stored in memory of the telecommunications device;
initiating the measurement and analysis application to activate measuring a glucose level;
transmitting a first signal to activate a measurement device placed on a human body through a wireless transmitter, the measurement device to measure the glucose level;
measuring the glucose level;
detecting a second signal received from the wireless transmitter connected to a measurement device, upon completion of measuring the glucose level;
receiving the glucose level from the measurement device to a wireless receiver;
comparing the received glucose level to a baseline glucose level predetermined by a user, the baseline glucose level identified as before a meal; and
visually presenting the received glucose level and a symbol on the user interface of the telecommunications device.

2. The method of claim 1, wherein the telecommunications device is a cellular device, a smart phone, a portable media player, a computing device, or a handheld computing device that operates in wireless networks such as a Personal Area Network (PAN), a Local Area Network (LAN), or a Wide Area Network (WAN).

3. The method of claim 1, wherein the initiating the measurement and analysis application to measure a glucose level comprises at least one of manual activation by receiving input on the user interface or automatic activation by identifying predetermined time periods to measure the glucose level.

4. The method of claim 1, further comprising:
presenting the received glucose level as a numerical number or on a graph within a predetermined range;
determining the symbol based on comparing the received glucose level to the baseline glucose level, when the received glucose is approximately about the same number as the baseline glucose level, the symbol is a smiley face, a thumbs up, or a color indicator; and
determining the symbol based on comparing the received glucose level to the baseline glucose level, when the received glucose is approximately 15 mg/dl less than or greater than the baseline glucose level, the symbol is a frown face, a thumbs down, or the color indicator.

5. The method of claim 1, further comprising:
determining whether the received glucose level is outside or within a glucose range predetermined by the user;
in response to the received glucose level being within the glucose range, storing the received glucose level in memory of the telecommunications device; and
in response to the received glucose level being outside the glucose range, generating an alert on the telecommunications device to vibrate, to play an audible sound, to vibrate and to play the audible sound, to send an email to an identified address, to send a text message to an identified cellular number, or to send the email to the identified address and to send the text message to the identified cellular number; and
storing the received glucose level in memory of the telecommunications device.

6. The method of claim 1, further comprising:
determining whether the received glucose level is within, below, or above a glucose range predetermined by the user;
in response to the received glucose level being within the glucose range, providing a recommendation for a course of action to take: continue physical activity without any adjustments;
in response to the received glucose level being below the glucose range, providing a recommendation for a course of action to take: to eat or to drink a fast-acting carbohydrate, to modify physical activity, or to adjust medication;
in response to the received glucose level being above the glucose range, providing the recommendation for the course of action to take: to adjust dietary selections, to modify physical activity, or to take medication;
presenting the received glucose level within, below, or above the glucose range in a chart; and
storing the received glucose level memory of the telecommunications device.

7. The method of claim 1, further comprising visually presenting a chart of received glucose levels for at least one of a 24 hour period, a week, a month, or a 3 month period.

8. The method of claim 1, further comprising saving received glucose levels as a file, the file saved on the telecommunications device, saved on a memory stick, attached to an email, or transferable to a computing device.

9. The method of claim 1, further comprising automatically saving received glucose levels for a predetermined time period and transmitting the saved received glucose levels to a medical provider; and
in response to the received glucose levels transmitted to the medical provider, receiving a medical treatment plan from the medical provider.

10. The method of claim 1, further comprising receiving input on the user interface to activate a logbook to record physical activity, dietary information, and medication dosage levels to correspond to received glucose levels.

11. A telecommunications device to communicate information in a network, the telecommunications device comprising:
a memory configured to store one or more applications with processor-executable instructions;
a processor configured to execute the one or more applications, an application to measure and to analyze a glucose level;
a communication interface (receiver) configured to transmit a signal to activate a measurement device placed on a human body, the measurement device to measure the glucose level;
the communication interface (receiver) configured to detect another signal and to receive the glucose level measured with a measurement device placed on the human body;
a functionality comparing module configured to compare the received glucose level to a baseline glucose level;
a functionality indicator module configured to associate an indication regarding the received glucose level compared to the baseline glucose level;
a user interface on a display of the apparatus configured to present the glucose level and the indication; and
the communication interface configured to send and to receive information regarding the received glucose level over the network.

12. The telecommunications device of claim 11, wherein the telecommunications device is a cellular device, a smart phone, a portable media player, a computing device, or a handheld computing device that operates in wireless networks such as a Personal Area Network (PAN), a Local Area Network (LAN), or a Wide Area Network (WAN).

13. The telecommunications device of claim 11, the application to measure the glucose level is configured to manually initiating the application by receiving input on the user interface or automatically activating the application by setting up predetermined time periods to measure the glucose level.

14. The telecommunications device of claim 11, further comprising processor-executable instructions configured to determine whether the received glucose level is outside or within a glucose range predetermined by a user;
in response to the received glucose level being outside the glucose range, generating an alert functionality on the telecommunications device to vibrate, to play an audible sound, to vibrate and to play the audible sound, to send an email to an identified address, to send a text message to an identified cellular number, or to send the email to the identified address and to send the text message to the identified cellular number;
storing the received glucose level in memory of the telecommunications device; and
in response to the received glucose level being within the glucose range, storing the received glucose level in memory of the telecommunications device.

15. The telecommunications device of claim 11, further comprising processor-executable instructions configured to presenting the received glucose level as a numerical number with the indication;
presenting the indication based on comparing the received glucose level to the baseline glucose level, when the received glucose is approximately about the baseline glucose level, the indication is a smiley face, or a green color indicator; and
presenting the indication based on comparing the received glucose level to the baseline glucose level, when the received glucose is outside the baseline glucose level, the indication is a frown face, a red color indicator, or a yellow color indicator.

16. The telecommunications device of claim 11, further comprising processor-executable instructions configured to:
associating the received glucose level with a physical activity, dietary information, or medication information based in part at least from a calendar or a logbook;
in response to the physical activity, dietary information, or medication information, determining whether the received glucose level is below or above a glucose range predetermined by a user;
in response to the received glucose level being below the glucose range, providing a recommendation for a course of action to take: to eat or to drink a fast-acting carbohydrate, to modify physical activity, to adjust medication, or to seek medical attention;
in response to the received glucose level being above the glucose range, providing the recommendation for the course of action to take: to adjust dietary selections, to modify physical activity, to take medication, or to seek medical attention; and
storing the received glucose level in memory of the telecommunications device.

17. The telecommunications device of claim 11, further comprising processor- executable instructions configured to:
automatically saving received glucose levels for a predetermined time period and transmitting the received glucose levels that have been stored to a medical provider; and
in response at least based in part on the received glucose levels transmitted to the medical provider, receiving a medical treatment plan from the medical provider.

18. One or more processor-readable storage media containing instructions that, when executed by a processor, perform acts comprising:
presenting a user interface on a display of a telecommunications device, the user interface including a measurement and analysis application stored in memory of the telecommunications device;
initiating the measurement and analysis application to activate measuring a biological fluid level;
transmitting the instructions to activate a measurement device placed on a human body to measure the biological fluid level;
measuring the biological fluid of the human body;
receiving the biological fluid level from the measurement device to a wireless receiver;
comparing the received biological fluid level to a baseline biological fluid level predetermined by a user; and
visually presenting the received biological fluid level on the user interface of the telecommunications device.

19. The one or more processor-readable storage media of claim 18, further comprising instructions to:
determining whether the received biological fluid level is outside or within a biological fluid range predetermined by the user;
in response to the received biological fluid level being outside the biological fluid range, generating an alert on the telecommunications device to vibrate, to play an audible sound, or to vibrate and to play the audible sound;
storing the received biological fluid level in memory of the telecommunications device; and
in response to the received biological fluid level being within the biological fluid range, storing the received biological fluid level in memory of the telecommunications device.

20. The one or more processor-readable storage media of claim 18, further comprising instructions to:
determining whether the received biological fluid level is below or above a biological fluid range predetermined by the user;
in response to the received biological fluid level being below the biological fluid range, providing a recommendation for a course of action to take: to eat or to drink a fast-acting carbohydrate, to modify physical activity, to adjust medication, or to seek medical attention;
in response to the received biological fluid level being above the biological fluid range, providing the recommendation for the course of action to take: to adjust dietary selections, to modify physical activity, to take medication, or to seek medical attention; and
storing the received biological fluid level in memory of the telecommunications device.

21. The one or more processor-readable storage media of claim 18, wherein the biological fluid is used to record a level for perspiration, salvia, heart rate, pulse, cholesterol, respiratory, or glucose.

22. The one or more processor-readable storage media of claim 18, further comprising instructions to:
measuring at least two or more biological fluid levels for different types of evaluations;
associating the two or more received biological fluid levels with a physical activity, dietary information, or medication information based in part at least from a calendar or a logbook;
in response to the physical activity, dietary information, or medication information, determining whether the received biological levels are below or above a biological fluid level range predetermined by a user;
in response to one of the received biological fluid levels being below the biological fluid range, providing a recommendation for a course of action to take: to modify physical activity, to adjust medication, or to seek medical attention;
in response to one of the received biological fluid levels being above the biological fluid range, providing the recommendation for the course of action to take: to adjust dietary selections, to modify physical activity, to take medication, or to seek medical attention; and
storing the received biological fluid levels in memory of the telecommunications device.
